(19) United States
(12) Patent Application Publication
(10) Pub. No.: US 2005/0080045 A1
(43) Pub. Date: Apr. 14, 2005

(54) Composition comprising dextrinsulfate for the treatment of sexual transmitted diseases (std)

(76) Inventor: Alan Chapman, Amersham (GB)

Correspondence Address: MYERS BIGEL SIBLEY & SAJOVEC, PO BOX 37428, RALEIGH, NC, 27627, US

(21) Appl. No.: 10/499,900

(22) PCT Filed: Dec. 24, 2002
(86) PCT No.: PCT/GB02/05926

(30) Foreign Application Priority Data
Dec. 24, 2001 (GB) 0130756.0
Mar. 13, 2002 (GB) 0205861.8
May 10, 2002 (GB) 0210735.7

(51) Int. Cl. A61K 31/715

(52) U.S. Cl. 514/58

(57) ABSTRACT

There is described a method of treating, alleviating or preventing the transmission of a sexually transmitted disease which comprises the topical administration of dextrin sulphate. There is also described a composition for use in the treatment, alleviation or prevention of a sexually transmitted disease which comprises dextrin sulphates in gel form.

This invention relates to pharmaceutically active materials and compositions and a novel method of treatment.

In particular the invention to novel formulations of dextrin sulphates and to the use of such materials and compositions as agents in the topical treatment of human sexually transmitted diseases (STDs).

It is known that some sulphated polysaccharides have anti-HIV activity; see, for example, European Patent Specification No. 0 240 098. This specification discloses highly sulphated oligosaccharides obtained by sulphation of dextrins of relatively low molecular weight.

It is known that dextrin sulphate has antilipaemic activity. U.S. Pat. No. 3,017,407 discloses antilipaemic agents comprising sulphated polysaccharides selected from the group consisting of corn starch dextrin and corn syrup solids containing an average of between about 5 and 15, and preferably between about 8 and 12 glucose units per molecule, containing between about 1.5 and 3, sulphate groups per molecule. There is no suggestion therein that any form of dextrin sulphate has activity against STDs.

Dextrin is a mixture of polymers of glucose and the glucose units may be substituted in one or more of the 2, 3 and 6 positions by sulphate groups. A dextrin sulphate of use in the present invention may have up to two sulphate groups per glucose unit.

European Patent No. 0 550 532 describes the use of dextrin sulphates in the treatment of human immunodeficiency virus (HIV).

Administration of dextrin sulphate in patients with AIDS may reduce the viral load of HIV-1. It is thought that three different mechanisms may operate: (i) binding of the drug to a cell surface protein on lymphocytes and MDM to block viral entry, (ii) inducing the release of MIP-1α and MIP-1β from tissue macrophages, which then block viral entry into CD4+ T lymphocytes and macrophages by binding to CCR-5, and (iii) an intracellular mechanism in tissue macrophages.

It has also been known for some time that dextrin sulphate gel is potentially useful as an intravaginal virucide (Stafford et al., 1997. J. Acquired Immune Deficiency Syndrome & Human Retrovirology 14: 213-218). The term “microbicide” is now preferred but is synonymous with virucide and vaginal microbicide (McCormack et al., 2001. British Medical J. 322: 410-413).

U.S. Pat. No. 6,063,773 describes a method of reducing the risk of infection and the risk of conception, which comprises administering an effective amount of a cellulose sulphate.

US Patent Application No. 2002/0151521, which is an intervening publication, describes compositions comprising an effective amount of a microbicidal agent to prevent sexually transmitted diseases and a hyaluronate. The compositions, and the microbicidal agents in particular, are described as being spermicidal. Whilst it is often desirable to combine microbicidal and spermicidal activity, there is a need for a non-spermicidal microbicidal agent.

We have now surprisingly found that dextrin sulphates are also useful in the treatment, alleviation or prevention of sexually transmitted diseases other than HIV when administered topically. Furthermore, dextrin sulphates exhibit little or no spermicidal activity and therefore offer, inter alia, an alternative method of reducing risk of infection whilst avoiding spermicidal effects.

Thus according to the invention we provide a method of treating, alleviating or preventing the transmission of a sexually transmitted disease (STD) which comprises the topical administration of dextrin sulphate, provided the sexually transmitted disease is not one caused by HIV.

The method of the invention especially provides a method of treating, alleviating or preventing the transmission of a sexually transmitted disease (STD) as hereinbefore described, wherein the method is substantially non-spermicidal.

A preferred method of the invention comprises preventing the transmission of an STD, provided the STD is not one caused by HIV.

We especially provide a method which comprises the administration of dextrin sulphate in, around or on the genitalia, the genito urinary tract and/or the rectum. Thus, the method of the invention may comprise intravaginal administration of dextrin sulphate, penile administration or rectal administration of dextrin sulphate.

The method of the invention may comprise the treatment of any known STD or combination of STDs, provided that the STD is not one caused by HIV. Thus, the STD may comprise a viral disease, provided it is not HIV, a bacterial disease, a protozoal disease or a fungal disease. In a preferred aspect of the invention the STD is a viral disease, other than HIV. We especially provide a method wherein the viral disease is genital herpes and particularly acyclovir resistant genital herpes.

In an alternative preferred aspect of the invention the STD is a fungal disease, such as Candida, e.g. Candida albicans. In a further alternative preferred aspect of the invention, the STD is a bacterial disease, such as chlamydia.

However, specific STDs which may be mentioned are bacterial vaginosis, chlamydia, genital herpes, genital warts, gonorrhoea, syphilis, trichomoniasis and Candida.

When the invention comprises the treatment alleviation or prevention of HPV, this may be manifested as the treatment, alleviation or prevention of genital warts.

The dosage of dextrin sulphate used may vary, depending upon, inter alia, the nature and severity of the disorder. However we have found that a suitable dosage comprises administering from 1 to 10 ml of a formulation comprising at least 1 μg/ml of dextrin sulphate, preferably from 1 μg/ml to 105 μg/ml, more preferably 500 μg/ml to 10⁵ μg/ml, most preferably 4 μg/ml, 1×10⁴ μg/ml (a 1% w/v solution), 2×10⁴ μg/ml (a 2% w/v solution) or 4×10⁴ μg/ml (a 4% w/v solution). Preferably, the method of the invention comprises administration of from 2 to 4 ml of a formulation as hereinbefore described, e.g. in gel form. The formulation may be administered at any time. However, the formulation of the invention is more efficacious when administered immediately before or shortly before-sexual activity although it will be understood that the formulation may be administered earlier.

A dextrin sulphate of use in the present invention may have up to two sulphate groups per glucose unit. Dextrin is a mixture of polymers of glucose and the glucose units may be substituted in one or more of the 2, 3 and 6 positions by sulphate groups.

A dextrin sulphate of use in the present invention may have up to two sulphate groups per glucose unit and preferred dextrin sulphates are those having about 1, or between 0.5 and 1.5, preferably up to 1.2, sulphate groups per glucose unit. More preferably, the agent is the 2- or 6-sulphate of dextrin or a mixture thereof.

Dextrin 3-sulphates may have relatively poor activity in the treatment of STDs, by comparison with dextrin 2- and 6-sulphates. It follows that for a given sulphate content the anti-STD activity of a dextrin sulphate may be inversely related to the proportion of 3-sulphation. Under most reaction conditions the 3-OH group of the glucose residue in a dextrin has been found to be less reactive than the 2-OH and 6-OH groups. Therefore, enhanced anti-STD activity per sulphate group can be achieved by keeping the degree of sulphation relatively low, thereby reducing the extent of 3-sulphation.

However, in selecting a particular sulphated dextrin as an anti-STD agent conflicting factors are encountered. Thus, generally speaking:—
1. For a given sulphate content:—
   (a) the toxicity increases with increasing molecular weight, and
   (b) the anti-STD activity increases with increasing molecular weight.
2. For a given molecular weight:—
   (a) the toxicity increases with increasing sulphate content, and
   (b) the anti-STD activity increases with increasing sulphate content.

It has seemed that dextrin sulphates might in fact be unusable in practice as anti-STD agents because satisfactory anti-STD activity appeared to go hand-in-hand with unacceptable toxicity, either because the molecular weight was too high or because the sulphate content was too high.

By restricting the degree of substitution to a maximum of 2 the present invention makes it possible to produce a dextrin sulphate having adequate anti-STD activity while keeping toxicity within acceptable limits. With a relatively low degree of substitution the proportion of 3-sulphation can be kept low, so that the toxicity imported into the dextrin sulphate by 3-substitution is avoided. If a dextrin is fully substituted, i.e. to give the 2,3,6-sulphate, one-third of the sulphate groups are 3-sulphate groups, which give rise to additional toxicity out of all proportion to the extent to which they enhance the anti-STD activity. The extent to which 3-sulphation occurs when the degree of substitution is kept below 2 varies with the nature of the sulphation process, but is normally substantially less than that of 2-sulphation or 6-sulphation. Presently available analytical techniques do not easily permit accurate analysis of the extent of sulphation at the three available sites, but an examination of the n.m.r. spectrum of a dextrin sulphate gives a sufficient indication of this for practical purposes. The total sulphate content can of course be evaluated by conventional analytical methods, normally by determining the sulphur content.

The molecular weight of dextrin sulphate of use in this invention may vary over a wide range. By way of example, dextrin sulphate of use in the present invention may have a weight average molecular weight of from 15,000 to 25,000 as determined on the dextrin used to prepare the dextrin sulphate. The technique used to determine molecular weight of the dextrin is high-pressure liquid chromatography, particularly gel permeation chromatography techniques (GPC), using chromatographic columns calibrated with dextran standards, as designated by Alsop et al, J Chromatography 246, 227-240 (1982); and/or other techniques known per se.

Dextrin sulphate can be prepared by first hydrolysing starch to produce dextrin which may then be sulphated to produce dextrin sulphate. For example, use of a trimethylamine/sulphur trioxide complex in aqueous alkaline medium gives predominantly the 2-sulphate. Treatment of dextrin with cyclamic acid in dimethylformamide gives the 6-sulphate. The 3-sulphate may be made by first acetylating dextrin, then sulphating it with trimethylamine/sulphur trioxide complex in dimethylformamide and finally removing the acetyl groups with aqueous sodium hydroxide.

It is preferred to use dextrin sulphate in which there is a low proportion of low molecular weight material. As has been mentioned above, dextrin is made by hydrolysis of starch, typically by treatment of various starches with dilute acids or with hydrolytic enzyme. Such methods produce glucose polymers with a wide range of polymerisation. The degree of polymerisation (D.P.) varies from one or two up to comparatively high numbers. The direct hydrolysis product of starch might contain up to 60% by weight of material having a D.P. less than 12. In a preferred aspect of the present invention, the dextrin derivative contains a relatively high proportion of glucose polymers of D.P. greater than 12. Preferably, the dextrin derivative contains at least 50% by weight of glucose polymers of D.P. greater than 12.

More preferably, the dextrin derivative contains less than 10% by weight of glucose polymers having a D.P. less than 12. Most preferably, the dextrin derivative contains less than 5% by weight of glucose polymers having a D.P. less than 12. Such dextrin derivatives are prepared from dextrin which has been fractionated to remove dextrin with a low D.P. Known fractionation techniques may be used, including solvent precipitation and membrane fractionation.

A method of preparing a glucose polymer mixture is described in Example 2 of GB 2154469. This mixture has a weight average molecular weight of 23,700 and contains 91.9% of polymers having a degree of polymerisation greater than 12 and 7.9% of polymers having a degree of polymerisation from 2 to 10.

It is also preferred that the dextrin derivative contains little or no material with a high molecular weight. More preferably, the dextrin derivative contains little or no material with a molecular weight greater than 40,000.

Dextrin sulphate may be effective in relatively low concentrations. Dextrin sulphate appears to be effective against STDs even with a relatively low degree of sulphation. For instance, a degree of sulphation as low as one sulphate group per glucose unit, or even lower, is found to be effective at relatively low concentrations. This has the advantage that the amount of sulphation can be kept to such a low level as to avoid the side effects and toxicity which might otherwise be experienced with highly sulphated materials.

The invention also provides an agent for use in the treatment of an STD, the agent being dextrin sulphate which contains at most 2 sulphate groups per glucose unit and contains at least 50% of polymers of a degree of polymerisation greater than 12.

According to a further feature of the invention we provide a composition for use in the treatment, alleviation or prevention of a sexually transmitted disease provided that the sexually transmitted disease is not one caused by HIV which comprises dextrin sulphates in gel form.

Thus the composition as hereinbefore described may comprise at least 1 μg/ml of dextrin sulphate, preferably from 1 μg/ml to 10⁵ μg/ml, more preferably from 500 μg/ml to 10⁵ μg/ml, most preferably 4 μg/ml, 1×10⁴ μg/ml (a 1% w/v solution), 2×10⁴ μg/ml (a 2% w/v solution) or 4×10⁴ μg/ml (a 4% w/v solution). The composition may preferentially be packaged in a single unit dosage form. Thus the composition may be made up in, for example a sachet or ampoule comprising from 1 to 10 ml of the composition, preferably 2 to 4 ml.

The composition of the invention may also comprise one or more medicaments known to be effective in the treatment, alleviation or prevention of a sexually transmitted disease. Preferably, when the composition of the invention includes such a medicament, the medicament is one which possesses some preventative efficacy or effectiveness. A variety of medicaments may be chosen. In particular, a group of antimycotic (antifungal) drugs known as azole derivatives have been found to be effective in the treatment of Candida species and Trichomonas infections. The main drugs used are; clotrimazole, miconazole, econazole, fenticonazole, fluconazole, itraconazole, tindazole and ketoconazole. They work by blocking production of ergosterol, the main sterol in the fungal cell membrane. This ultimately affects the action of membrane-associated enzymes so that replication of the fungus is inhibited. It also prevents the non-pathogenic form of the microbe developing into the invasive form (hyphae). The drugs produce cell necrosis by inhibiting peroxidase enzymes. Development of resistance to the imidazoles is rare.

Clotrimazole interferes with amino acid transport into the organism by attacking the cell membrane. It is a drug of choice for treatment of candidiasis of the vagina. Adverse effects can occur on application to the skin and include stinging, erythema, pruritis, peeling. The incidence however is low. Intravaginal administration can be associated with a burning sensation and lower abdominal cramps. This drug is available as a cream or tablet for intravaginal use and can also be applied topically.

Miconazole is used topically, but rarely systemically due to toxicity issues. Systemic use is by intravenous infusion and is associated with fever, nausea, and a rash. It also can enhance the action of oral anticoagulants leading to haemorrhage. Around 15% of C. albicans are resistant to clotrimazole and miconazole. Recurrent infections can be treated with fluconazole.

Econazole is available for topical application. Adverse effects include burning and itching sensations, though these have been observed in just 3% of users. Less than 1% of topical econazole is absorbed. A 1% w/v cream is applied twice a day for 2 weeks.

Ketoconazole can be administered orally to treat superficial mycoses. The toxicity associated with the drug means that its use is only advised for those mycoses which have not responded to topical agents.

Other treatments include nystatin, a polyene antibiotic. It is used solely to treat candidiasis by topical, intravaginal and oral routes. Significant absorption has not been observed with any of these routes. Intravaginal application of the drug is by administration of a vaginal tablet. Dosage ranges between 100,000-200,000 units daily for a period of two weeks. Adverse effects reported by Lehne et al are limited to oral nystatin (occasionally causes gastrointestinal disturbances) and topical application (can cause local irritation).

Treatment of bacterial vaginosis usually consists of metronidazole or clindamycin by mouth or intravaginally. Metronidazole is also used in the treatment trichomonas infections. Local sulphonamide antibiotics are also indicated for bacterial vaginosis.

Antivirals such as acyclovir and famcyclovir (and the acyclovir precursor valacyclovir), which cause selective inhibition of DNA synthesis in virus infected cells only, are used in the treatment and long term suppression of genital warts. Other topically-applied treatments include the immune response modifier, imiquimod and the cytotoxic, podophyllotoxin.

According to a further aspect of the invention we provide a method of treating, alleviating or preventing the transmission of a sexually transmitted disease (STD) which comprises the topical administration of a substantially non-spermicidal gel. In this aspect of the invention, we especially provide a method as hereinbefore described wherein the sexually transmitted disease is not one caused by HIV.

Further, the invention provides a composition comprising the dextrin sulphate composition as hereinbefore described, together with an inert carrier or diluent.

It is within the scope of this invention for the dextrin sulphates to be provided in powder form such that the powder may be admixed with an appropriate solvent or diluent to form a gel.

The composition of the invention may also include a viscosity adjusting agent, e.g. a viscosity reducing agent. Thus, the use of such an agent allows, for example, a higher concentration of dextrin sulphate to be used whilst maintaining the viscosity of the material, e.g. a gel. A variety of such agents may be used, however a preferred such agent is a polymer, such as a Carbopol®. The concentration of the viscosity improving agent may vary, depending, inter alia, upon the nature of the material used. However, a preferred concentration is from 0.5 to 5% w/v. Most preferably, the viscosity improving agent is a Carbopol® which is present as 1% w/v or 4% w/v of the composition.

When the dextrin sulphate is made up in to a gel it may be administered directly to the vagina, rectum or penis. Alternatively, the dextrin sulphate gel may be administered using conventionally known prophylactic devices. A preferred prophylactic device is a conventionally known condom, e.g. a condom which may be coated, either internally or externally with a dextrin sulphate gel.

We further provided the use of dextrin sulphates in the manufacture of a topically administrable composition for the treatment, alleviation or prevention of a sexually transmitted disease (STD), provided that the STD is not one caused by HIV.

The use as hereinbefore described preferably comprises the prevention of an STD.

In the use of this aspect of the invention the STD is preferably a viral disease, other than HIV, e.g. HPV or genital herpes and especially acyclovir resistant genital herpes.

In a further aspect of the invention, the use as hereinbefore described may comprise manufacture of a topically administrable composition for the treatment, alleviation or prevention of a sexually transmitted disease (STD) wherein the STD is a fungal disease, e.g. Candida, such as Candida albicans.

In an alternative aspect of the invention, the use as hereinbefore described may comprise the manufacture of a topically administrable composition for the treatment of a bacterial disease, such as chlamydia.

The method of the invention is advantageous in that, inter alia, the dextrin sulphate has little or no spermicidal activity, whilst still possessing the desired microbicidal activity.

The invention will now be illustrated with reference to the following examples and drawings, in which FIG. 1 is an illustration of the microbicidal activity of dextrin sulphate against bovine papillomavirus-1 (BPV-1).

### EXAMPLE 1

Preparation of Dextrin 3-Sulphate

16.2 of the aforementioned dextrin of Example 2 of GB 2,154,469 in dimethylformamide (150 ml) was stirred and heated until dissolved, then cooled to ambient temperature. Acetic anhydride (23 ml, 0.24 mole) was added slowly with stirring. A transient precipitation occurred when this had redissolved, triethylamine (25 ml, 0.18 mole) was added and the mixture stirred for 2 days. The solution was then poured in a thin stream with stirring into water (700 ml), the precipitate was filtered off, washed with water and dried to give 23 g of white powder.

The acetylated dextrin (12.3 g) in dimethylformamide (75 ml) was stirred until dissolved then trimethyl amine sulphur trioxide complex (15 g) was added and the mixture was stirred at ambient temperature overnight. Further trimethylamine sulphur trioxide (10 g) was added and the mixture heated at 60 C for 3 hours. The solution was cooled and poured into acetone (500 ml) to give a sticky residue. The supernatant was decanted and the residue kneaded with fresh acetone (50 ml) and then the supernatant decanted. The residue was dissolved in water (150 ml) and the remaining acetone stripped off under vacuum. A solution of NaOH (5 g) in water (10 ml) was added giving trimethylamine gas. The strongly basic solution was stored for 2 h, dialysed against water for 4 days and freeze dried, to give 10.2 g. The I.R. spectrum showed peaks for acetate (1750 CM¹) and sulphate (1240 CM¹). The product (10 g) was redissolved in water (150 ml) and NaOH (1 g) in water added and the mixture stirred 3 h at ambient temperature. The solution was poured into ethanol (300 ml), the supernatant was decanted and the sticky residue kneaded with fresh ethanol (150 ml) to give a solid. The solid was filtered off, washed with methanol and dried to a brown powder. The powder was dissolved in water (200 ml) and decolourising charcoal (5 g) added. The solution was warmed then filtered twice and freeze-dried to give 7.2 g, sulphate, 46.9%.

### EXAMPLE 2

Preparation of Dextrin 6-Sulphate

10 g of the same dextrin as in Example 1 in dimethylformamide (100 ml) was heated and stirred at 78° C. When the dextrin had all dissolved cyclamic acid (22.5 g) was added and the reaction continued for 1.5 h. A solution of NaOH (5 g) in water (5 ml) and ethanol (50 ml) was added and the mixture poured into diethyl ether (400 ml). The solid was filtered off, washed with ether and air dried. The solid was dissolved in water (100 ml), sodium acetate (50 g) added and the solution dialysed against water for 4 days then freeze dried to give 15.4 g, sulphate 47.2%.

### EXAMPLE 3

Preparation of Dextrin 2-Sulphate

40 g of the same dextrin as in Example 1 in distilled water (150 ml) were stirred in a round bottomed flask at 30° C. When the dextrin had all dissolved trimethylamine sulphur trioxide (51 g) were added to the solution. The reaction mix was stirred for thirty minutes. Sodium hydroxide (62.5 ml @ 40% w/v) was added dropwise to the reaction mix over a period of one hour. The reaction mix was then stirred for a further two hours and filtered under vacuum. The resultant solution was dialysed for one day against tap water and one day against distilled water. The dialysed solution was then concentrated by evaporation at reduced pressure. The concentrated solution contained 30 g of dissolved solids at 36% w/w (wrt dry solids) sulphate.

The products of Examples 1, 2 and 3 have been identified as the 3-,6- and 2-sulphates respectively by examination of their n.m.r. spectra.

The 13C n.m.r. spectrum of the original dextrin shows six lines. These can mostly be assigned, by reference to standard compounds, as: 100.3, C-1; 77.6, C-4; 73.9, C-3; 72.2 and 71.8, C2 and C-5; 61.1, C-6.

The n.m.r. spectra of both the 3- and 6-sulphates of glucose have been reported (S. Honda, Y. Yuki and K. Tahiura, Carbohydrate Research (1973) Volume 28, pages 130 to 150) and compared to the free sugars. Thus, 3-O-sulphation was observed to cause 8.5 or 9.5 ppm downfield shift for C-3, a 1.1 ppm upfield shift for C-2 and 2.2 ppm upfield shift for C-4, but little change for other positions. For 6-O-sulphation, a downfield shift of 6.2 ppm was observed for C-6 and upfield shifts of 1.7 ppm for C-5 and 0.3 ppm for C-4, with little change in other positions.

The n.m.r. spectrum of the product of Example 1 shows a strong signal at 61.1 ppm, characteristic of unmodified C-6-OH. Prominent new signals have appeared at 82.2 and 82.5 ppm. These are close to the chemical shift of 82.7 ppm reported for C-3 in D-glucose-3-sulphate and are therefore assigned to dextrin-3-sulphate. This assignment is supported by the virtual disappearance of the signal at 77.6 ppm in the original dextrin for C-4. Substitution at 0-3 is expected to cause an upfield shift of the signal for C-4, taking it under the envelope of other signals. New peaks at 70.2 and 70.8 ppm are attributed to C-2 in a 3-sulphate by upfield shift from the original position at 72.2 or 71.8 ppm. The C-1 region shows six closely spaced lines between 100.1 and 98.3 ppm slightly upfield from that in the original dextrin. From this data it appears that the product of Example 1 is sulphated almost entirely in the 3-position.

The n.m.r. spectrum of the product of Example 2 shows that the original C-6 peak at 61.1 ppm has greatly diminished and new peaks have appeared at 67.5 ppm and 69.3 ppm, for C-6-O-sulphate (6.4 ppm downfield shift) and for C-5 adjacent to 6-O sulphate (2.5 or 2.9 upfield shift) respectively. This data indicates that the product of Example 2 is substituted primarily in the 6-position.

The n.m.r. spectrum of the product of Example 3, in comparison with that of the original dextrin, shows a major signal for unsubstituted C-6-OH at 61.1 ppm, unperturbed C-4 signal at 78.1 ppm, indicating free 3-OH and the major C-1 signal moved upfield to 99.8 ppm from its original position at 100.3 ppm. From this data it appears that the product of Example 3 is substituted primarily in the 2-position.

### EXAMPLE 4

In vitro Activity of Dextrin Sulphate on the Bovine Papillomavirus (BPV-1) Focus-Forming Assay

Introduction

Genital human papillomavirus (HPV) infections represent one of the most frequent sexually transmitted diseases (STDs). Although most infections spontaneously resolve within a year, a proportion of persistent infections can progress to invasive cervical cancer. To date, very few reagents with microbicidal activity against HPV infections have been described. These include reagents that specifically target HPVs such as monoclonal antibodies with virus neutralising activity, and virus non-specific agents such as povidone-iodine, alkyl sulphates and monocaprin. Several reagents that have microbicidal activity against a broad range of STDs have proven to be ineffective against papillomaviruses. Some of these agents also induce significant cellular cytotoxicity at high concentrations.

This experiment was conducted to determine the papillomavirus microbicidal activity of dextrin sulphate; a sulphated glucose polymer that does not show cellular cytotoxicity even at high concentrations (NB The term microbicide, rather than virucide, is used).

Experimental

Under normal conditions, the mouse cell line C127-D10 (a clone of the mouse cell line C127) grows to a confluent, contact-inhibited monolayer.

When infectious bovine papillomavirus-1 (BPV-1) is added to the C127-D10 cell line, BPV-1 causes transformation of the cells into a focus of cells that show altered morphology and which have lost, for example, contact inhibition features. Foci numbers are directly related to the number of BPV-1 virions added to the culture, and are a quantitative assay of the effect of BPV-1.

Aliquots of BPV-1, were diluted to give approximately 100-200 focus-forming units, were pre-incubated with dilutions of dextrin sulphate at concentrations ranging from 1 ng/ml to 20 mg/ml for 10 minutes at 37° C. prior to addition to mouse C127-D10 cells. The culture was incubated for two weeks, with media changes every three to four days. Foci were enumerated following staining of the monolayer with crystal violet, and counted.

Results

The highest concentration of dextrin sulphate that was exposed to the cell line was 1 mg/ml. No toxicity was observed.

The dose at which 50% inhibition of foci formation was observed (ID50) was 10 μg/ml.

Previous experiments have shown correspondence between activity against BPV-1 and human papillomavirus type 11 (HIV-11).

The results are illustrated in FIG. 1: y-axis=mean number (±SD) of foci; x-axis=dextrin sulphate concentration (μg/ml); control cultures had a total of approximately 80 foci.

The data indicate that dextrin sulphate may be a useful non-toxic microbicidal compound that is active against a variety of STD agents including papillomaviruses.

### EXAMPLE 5

In vitro Activity of Dextrin Sulphate Against Chlamydia trachomatis (Cell Culture Assay)

Introduction

Chlamydia is a common sexually transmitted disease that is caused by the bacterium Chlamydia trachomatis. Because approximately 75% of women and 50% of men have no symptoms, most people infected with chlamydia are not aware of their infections and therefore may not seek health care. Untreated, chlamydia can cause pelvic inflammatory disease in women and urethral infection in men. Recent research has shown that women infected with chlamydia have a 3-5 fold increased risk of acquiring HIV, if exposed.

Chlamydia can be treated and cured with antibiotics. A single dose of azithromycin or a week of doxycycline (twice daily) are the most commonly used treatments. Safe sexual behaviour and prevention of infection are to be preferred, however, owing to prevalence (chlamydia is one of the most frequently reported infectious diseases), and hence cost, and the seriousness of sequelae if untreated.

Experimental

McCoy cell monolayers were prepared on coverslips in plastic Bijoux. Cells were infected with Chlamydia trachomatis either alone or in the presence of dextrin sulphate. The Sa₂f strain of C. trachomatis was used. Sa₂f, which causes lymphogranuloma venereum, is similar to C. trachomatis. It is a heavily passaged laboratory strain which can infect McCoy cells without the need for spinning (for most strains of C. trachomatis, it is necessary to spin the cells onto the monolayers in the presence of cyclohexamide in order for infection of the monolayers to occur).

After incubation, coverslips were fixed and stained for C. trachomatis. The number of inclusion bodies per coverslip was estimated by fluorescence microscopy.

Experimental and Results

Details are given in Table I.

### Conclusion

The data indicate that dextrin sulphate may be useful microbicidal compound that is active against a variety of STD agents including Chlamydia.

TABLE I

| Expt. | Inhibition Experimental | Results |
|---|---|---|
| 2.1 | with preincubation: | |
| 2.1.1 | Sa₂f preincubated with dextrin sulphate | inhibition by dextrin |
| | before infecting the cell line | sulphate |
| 2.1.2 | dextrin sulphate preincubated with the | inhibition by dextrin |
| | cell line before infection with Sa₂f | sulphate |
| 2.2 | Sa₂f and dextrin sulphate without | inhibition by dextrin |
| | preincubation | sulphate (50% inhibition |
| | | with 5-10 μg/ml) |
| 2.3 | use of different infecting doses of Sa₂f: | inhibition by dextrin |
| | | sulphate |
| 2.3.1 | larger infecting doses of Sa₂f | a) inhibitory activity |
| | (400 inclusion bodies in 30 fields) | plateaued off |
| | | b) 100% inhibition could |
| | | not be achieved |
| | | c) 60% inhibition with |
| | | dextrin sulphate |
| | | 100 μg/ml |
| 2.3.2 | lower infecting doses (40 inclusion | a) inhibitory activity |
| | bodies in 30 fields) | plateaued off |
| | | b) 80-90% inhibition |
| | | with dextrin sulphate |
| | | 1000 μg/ml |
| 2.4 | use of the spinning step | inhibition by dextrin |
| | | sulphate (50% inhibition |
| | | with 5 μg/ml) |

### EXAMPLE 6

In vitro Activity of Dextrin Sulphate Against Herpes Simplex Virus (HSV).

Dextrin sulphate was added to the cell culture and then removed following an incubation period. Virus was then added as it is thought that dextrin sulphate attaches to cell receptors, thereby blocking subsequent viral entry. Following incubation with virus, dextrin sulphate in carboxylmethyl cellulose was added. This medium was used to form a gel layer to prevent any intracellular virus (that has bypassed the dextrin sulphate-cell receptor blocking interaction) from further infecting other cells.

Assay Procedure

(i) Seed 24 well cell culture plates with Vero cells (2×10⁵ cells/ml/well). Prepare one plate per virus plus 2 plates for controls.
(ii) Grow cells to confluency overnight at 37° in 5% CO₂.
(iii) Dilute virus as calculated to give about 100 plaques per well, a minimum volume of 5 ml per plate.

Plate Layout. Dextrin sulphate (D.S.) concentrations are in mg per ml.

The following table is a schematic representation of the “wells” on a agar plate.

| D.S. | D.S. | D.S. | D.S. | D.S. | Virus |
|---|---|---|---|---|---|
| 80 | 8 | 0.8 | 0.08 | 0.008 | control |
| D.S. | D.S. | D.S. | D.S. | D.S. | Virus |
| 80 | 8 | 0.8 | 0.08 | 0.008 | control |
| D.S. | D.S. | D.S. | D.S. | D.S. | Cell |
| 80 | 8 | 0.8 | 0.08 | 0.008 | control |
| D.S. | D.S. | D.S. | D.S. | D.S. | Cell |
| 80 | 8 | 0.8 | 0.08 | 0.008 | control |

(iv) Pipette cell medium off all wells except the cell control wells.
(v) Inoculate the appropriate well with 1 ml Dextrin sulphate made up in Minimum Essential Medium as per above layout.
(vi) Incubate the plate at 37° C. in CO₂ incubator for 1 hour.
(vii) Remove Dextrin sulphate.
(viii) Inoculate 200 μl of virus dilution per well.
(ix) Incubate the plate at 37° C. in CO₂ incubator for 1 hour.
(x) Add 1 ml Dextrin sulphate in CMC to appropriate wells, incubate the plates at 37° C. in CO₂ incubator for 60 hours.
(xi) Fix with 10% formalin in PBS by topping up the wells, leave for a minimum of 1 hour.
(xii) Decant formalin, rinse plate in water, stain with crystal violet for 5-10 mins, rinse and drain. Leave plates to dry before counting.

(xiii) Count plaques, calculate IC₅₀ by Grafit.

Results of HSV Susceptibility to Dextrin Sulphate
IC₅₀ (mg/ml)

| | | |
|---|---|---|
| HSV-1 | ACV sensitive | 1.13 |
| HSV-1 | ACV resistant | 2.77 |
| HSV-2 | ACV sensitive | 0.11 |
| HSV-2 | ACV resistant | 0.004 |
| SC-16 | Laboratory HSV-1 ACV sensitive | 0.33 |
| DM-21 | Laboratory HSB-1 ACV resistant | 0.91 |

Values mean of two determinations.

### EXAMPLE 7

In vitro Activity of Dextrin Sulphate Against Candida.

Materials and Methods

TABLE 1
Test and control strains and their sources

| Organism | Designation | | Source |
|---|---|---|---|
| Candida parapsilosis | NCPF 3938 | Control | NCPF |
| Candida krusei | NCPF 3953 | Control | NCPF |
| Candida albicans | ATCC 90028 | Test | ATCC |
| Candida parapsilosis | ATCC 22019 | Test | ATCC |
| Candida glabrata | MRC 7051586 | Test | Clinical isolate |
| Candida tropicalis | MRC 22682 | Test | Clinical isolate |
| Candida guillermondii | MRC 9967063 | Test | Clinical isolate |
| Candida albicans | MRC 7650467 | Test | Clinical isolate |
| Candida lusitaniae | MRC 993387 | Test | Clinical isolate |

All the organisms tested were from the National Collection of Pathogenic Fungi (NCPF), the American Type Culture Collection (ATCC) or from the Mycology Reference Centre culture collection (MRC). The organisms were routinely maintained on Sabouraud agar (Oxoid), with yeasts incubated at 37° C.

The sensitivity testing method used a microtitre plate format, with RPMI 1640 (Sigma) as the culture medium. Dextrin sulphate was dissolved in RPMI 1640, and a range of doubling dilutions was prepared in RPMI 1640 and dispensed into the microtitre plate. Dextrin sulphate was tested over the range 0.5-250 mg/ml. It was not possible to test higher concentrations due to solubility problems.

To prepare the inoculum for the test, the yeast test isolates were freshly subcultured and incubated overnight. The yeast inoculum consisted of a suspension with a spectrophotometric transmission of 85-90% at 530 nm. This was then diluted in RPMI-1640 to obtain a suspension of 2.5×10³ colony forming units per ml.

All test plates contained a number of controls. The medium only column checked for contamination and a medium and inoculum column was the 100% growth control. For each batch of plates tested, an azole sensitive and an azole resistant strain was included. Plates were incubated at 37° C. for 48 hours. Each organism was tested in duplicate to the test range of dextrin sulphate concentrations.

After incubation, plates were examined visually. The medium and inoculum column was taken as 100% growth and the other wells were compared to this. The Minimum Inhibitory Concentration (MIC₈₀) was taken as the first well with an 80% inhibition of growth (i.e. a residual 20% growth in comparison to the 100% growth well). Each plate was read independently by two observers.

1. A method of treating, alleviating or preventing the transmission of a sexually transmitted disease (STD) which comprises the topical administration of dextrin sulphate, provided the sexually transmitted disease is not one caused by HIV.

2. The method according to claim 1 characterized in that the method is substantially non-spermicidal.

3. The method according to claim 1 characterized in that the method comprises the prevention of an STD.

4. The method according to claim 1 characterized in that the method comprises administration of dextrin sulphate in, around or on the genitalia, the genito urinary tract and/or the rectum.

5. The method according to claim 4 characterized in that the method comprises intravaginal administration, penile administration or rectal administration of dextrin sulphate.

6. The method according to claim 1 characterized in that STD is selected from a viral disease, a bacterial disease, a protozoal disease, a fungal disease or a combination thereof.

7. The method according to claim 6 characterized in that the STD is a viral disease, other than HIV.

8. The method according to claim 7 characterized in that the viral disease is genital herpes or human papillomavirus (HPV).

9. The method according to claim 8 characterized in that the genital herpes is acyclovir resistant genital herpes.

10. The method according to claim 6 characterized in that the STD is a bacterial disease.

11. The method according to claim 10 characterized in that the bacterial disease is chlamydia.

12. The method according to claim 6 characterized in that the STD is a fungal disease.

13. The method according to claim 12 characterized in that the fungal disease is Candida.

14. The method according to claim 6 characterized in that the STD is bacterial vaginosis, chlamydia, genital herpes, genital warts, gonorrhoea, syphilis, trichomoniasis or a combination thereof.

15. The method according to claim 1 characterized in that the concentration of dextrin sulphate administered is at least 1 μg/ml.

16. The method according to claim 15 characterized in that the concentration of dextrin sulphate administered is in a range from 1 μg/ml to 10⁵ μg/ml.

17. (canceled)

18. The method according to claim 15 characterized in that the concentration of dextrin sulphate administered is 4 μg/ml, 1×10⁴ μg/ml, 2×10⁴ μg/ml or 4×10⁴ μg/ml.

19-22. (canceled)

23. The method according to claim 15 characterized in that the volume of dextrin sulphate administered is in a range from 1 to 10 ml.

24. The method according to claim 23 characterized in that the volume of dextrin sulphate administered is in a range from 2 to 4 ml.

25-27. (canceled)

28. The method according to claim 1 characterized in that the dextrin sulphate is also provided with a medicament known to be effective in the treatment, alleviation or prevention of a sexually transmitted disease.

29. The method according to claim 28 characterized in that the medicament is effective in the prevention of an STD.

30. The method according to claim 1 wherein there is at most two sulphate groups per glucose unit.

31. The method according to claim 30 wherein there is a range of 0.5 and 1.5 sulphate groups per glucose unit.

32. The method according to claim 31 wherein there is up to 1.2 sulphate groups per glucose unit.

33. The method according to claim 1, wherein the agent is dextrin 2-sulphate, dextrin 6-sulphate or a mixture thereof.

34. The method according to claim 1, wherein the dextrin sulphate contains at least 50% of glucose polymers of degree of polymerization greater than 12.

35. The method according to claim 1, wherein the dextrin sulphate contains less than 10% by weight of glucose polymers having a degree of polymerization less than 12.

36. The method according to claim 35 wherein the dextrin sulphate contains less than 5% by weight of glucose polymers having a degree of polymerization less than 12.

37. The method according to claim 1 wherein the weight average molecular weight of the dextrin sulphate is from 15,000 to 25,000.

38. The method according to claim 1, wherein the dextrin sulphate is substantially free from polymers of molecular weight greater than 40,000.

39. The method according to claim 1 wherein the dextrin sulfate is combined with an inert carrier or diluent.

40. The method according to claim 1 characterized in that the dextrin sulphate is in the form of a gel.

41. The method according to claim 40 characterized in that the gel is administered directly to the vagina, rectum or penis.

42. The method according to claim 41 characterized in that the dextrin sulphate gel is applied to a prophylactic.

43. The method according to claim 42 characterized in that the prophylactic is a condom.

44. A method of treating, alleviating or preventing the transmission of a sexually transmitted disease (STD) which comprises the topical administration of a substantially non-spermicidal gel.

45. A composition for use in the treatment, alleviation or prevention of a sexually transmitted disease which comprises dextrin sulphates in gel form provided that the sexually transmitted disease is not one caused by HIV.

46. The composition according to claim 45 characterized in that the concentration of dextrin sulphate is at least 1 μg/ml.

47. The composition according to claim 46 characterized in that the concentration of dextrin sulphate is from 1 μg/ml to 10⁵ μg/ml.

48. (canceled)

49. The composition according to claim 46 characterized in that the concentration of dextrin sulphate administered is 4 μg/ml, 1×10⁴ μg/ml, 2×10⁴ μg/ml or 4×10⁴ μg/ml.

50-53. (canceled)

54. The composition according to claim 46 characterized in that the dosage of dextrin sulphate is in a range from 1 to 10 ml.

55. The composition according to claim 54 characterized in that the dosage of dextrin sulphate is in a range from 2 to 4 ml.

56. The composition according to claim 45 characterized in that the composition also comprises one or more medicaments known to be effective in the treatment, alleviation or prevention of a sexually transmitted disease.

57. The composition according to claim 56 characterized in that the medicament is an antimycotic.

58. The composition according to claim 57 characterized in that the antimycotic is an azole derivative.

59. The composition according to claim 58 characterized in that the azole derivative is clotrimazole, miconazole, fluconazole, econazole, fenticonazole, itraconazole, tindazole, metronidazole, ketoconazole or a combination thereof.

60. The composition according to claim 56 characterized in that the medicament is an antibiotic.

61. The composition according to claim 60 characterized in that the medicament is a sulphonamide, nystatin or a combination thereof.

62. The composition according to claim 56 characterized in that the medicament is an antiviral, an immune response modifiers a cytotoxic or a combination thereof.

63. The composition according to claim 45 wherein the dextrin sulphate contains at most 2 sulphate groups per glucose unit and contains at least 50% of polymers of a degree of polymerization greater than 12.

64. The composition according to claim 63 wherein there are between 0.5 and 1.5 sulphate groups per glucose unit.

65. The composition according to claim 64 wherein there are up to 1.2 sulphate groups per glucose unit.

66. The composition according to claim 65 where the dextrin sulphate is dextrin 2-sulphate, dextrin 6-sulphate or a mixture thereof.

67. The composition according to claim 65 wherein the dextrin sulphate contains less than 10% by weight of glucose polymers having a degree of polymerization less than 12.

68. The composition according to claim 67 wherein the dextrin sulphate contains less than 5% by weight of glucose polymers having a degree of polymerization less than 12.

69. The composition according to claim 45 wherein the weight average molecular weight of the dextrin sulphate is in a range from 15,000 to 25,000.

70. The composition according to claim 69 wherein the dextrin sulphate is substantially free from polymers of molecular weight greater than 40,000.

71. The composition according to claim 45 characterized in that the dextrin sulphate gel is applied to a prophylactic.

72. The composition according to claim 45 wherein the dextrin sulphate is in admixture with an inert carrier or diluent.

73. A composition for use in the treatment, alleviation or prevention of a sexually transmitted disease, provided that the sexually transmitted disease is not one caused by HIV, which comprises dextrin sulphate in powder form such that the powder may be admixed with an appropriate solvent or diluent to form a gel.

74. The composition according to claim 45 wherein the composition includes a viscosity adjusting agent.

75. The composition according to claim 74 wherein the viscosity adjusting agent is a viscosity reducing agent.

76. The composition according to claim 74 wherein the viscosity adjusting agent is a polymer material.

77. The composition according to claim 76 wherein the polymer material is a Carbopol®.

78. The composition according to claim 74 wherein the viscosity adjusting agent is present in the composition at a concentration in a range from 0.5 to 5% w/v.

79. The composition according to claim 78 wherein the viscosity adjusting agent is a Carbopol® which is present as 1% w/v

80. The composition according to claim 78 wherein the viscosity adjusting agent is a Carbopol® which is present as 4% w/v.

81. A prophylactic condom characterized in that the condom is coated with dextrin sulphate.

82. The use of dextrin sulphate in the manufacture of a topically administrable composition for the treatment, alleviation or prevention of a sexually transmitted disease (STD), provided that the STD is not one caused by HIV.

83. The use according to claim 82 wherein the composition is suitable for the prevention of an STD.

84. The use according to claim 83 characterized in that the STD is a viral disease, other than HIV.

85. The use according to claim 84 characterized in that the viral disease is genital herpes or HPV.

86. The use according to claim 85 characterized in that the genital herpes is acyclovir resistant genital herpes.

87. The use according to claim 82 characterized in that the STD is a fungal disease.

88. The use according to claim 87 characterized in that the fungal disease is Candida.

89. The use according to claim 82 characterized in that the STD is a bacterial disease.

90. The use according to claim 89 characterized in that the bacterial disease is chlamydia.

91. The use according to claim 82 characterized in that the STD is bacterial vaginosis, chlamydia, genital herpes, genital warts, gonorrhoea, syphilis, trichomoniasis or combinations thereof.

92. (canceled)
